(19) United States
(12) Patent Application Publication
(10) Pub. No.: US 2010/0136068 A1
(43) Pub. Date: Jun. 3, 2010

(54) DERMATOLOGICAL COMPOSITION FOR PREVENTION AND/OR TREATMENT OF ROSACEA, OF COUPEROSE OR OF SKIN WHICH EXHIBITS DIFFUSE REDNESS OR SMALL DILATED VESSELS

(76) Inventors: Valerie Perier, Frouzines (FR); Stéphanie Rinaldin, Belleserre (FR)

Correspondence Address: BIRCH STEWART KOLASCH & BIRCH, PO BOX 747, FALLS CHURCH, VA, 22040-0747, US

(21) Appl. No.: 12/597,096

(22) PCT Filed: Apr. 23, 2008
(86) PCT No.: PCT/EP2008/054956

§ 371 Date: Oct. 22, 2009

(30) Foreign Application Priority Data
Apr. 23, 2007 (FR) 0754639

(51) Int. Cl. A61K 8/02; A61K 8/33; A61Q 17/04; A61P 17/00

(52) U.S. Cl. 424/401; 424/60

(57) ABSTRACT

The invention relates to a dermatological composition for the prevention and/or treatment of rosacea, of couperose or of skin which exhibits diffuse redness or small dilated vessels, characterized in that it contains: —at least one interference pigment comprising titanium dioxide-coated mica, transmitting a colour complementary to red; —at least one sunscreen that is active in the UVA and UVB ranges; —one or more soothing and/or moisturizing active ingredients; and —the rest as dermatologically acceptable excipient(s) necessary for formulating said composition.

The invention relates to a dermatological composition for preventing and/or treating rosacea, couperose or skins which exhibit diffuse red patches or small dilated vessels.

Couperose is a permanent red condition of convex areas of the face (nose, cheeks, forehead, chin . . . ), with sometimes a few vessels visible to the naked eye.

Rosacea is the whole of the outward signs (spots, patches, irritation and red patches of the eyes . . . ) which complicate couperose.

Physiopathology of rosacea is still poorly understood even if primitive vascular abnormality of the face is suspected. A cold climate, working in the heat and exposure to the sun are incriminated in the triggering of initial forms of rosacea.

Most anti-redness dermatological formulations resort to pigments with high hiding power which mechanically act by covering the abnormal red areas of the skin. However, these formulations are not satisfactory because they leave an unsightly opaque film on the skin. Other formulations use colored pigments which act by complementarity with red. However these formulations have the drawback of coloring the areas of the face which are not affected by red patches.

The inventors of the present patent application have developed a formulation with which it is possible to obtain an immediate effect of hiding red patches of the skin without having the drawbacks listed above on the one hand, and with which on the other hand, it is possible to prevent and treat rosacea in the short, medium and long term.

The object of the present invention is a dermatological composition for preventing and/or treating rosacea, couperose or skins which have diffuse red patches or small dilated vessels, characterized in that it contains:
at least one interference pigment comprising mica coated with titanium dioxide, transmitting a color which is complementary to red;
at least one sunscreen active in the UVA and UVB ranges;
one or several soothing and/or moisturizing active ingredients; and
   the balance as dermatologically acceptable excipient(s) required for formulating said composition.

The dermatological composition according to the present invention is provided with a triple action staged over time, i.e.:
immediate action of optically masking the red patches;
a soothing effect and/or protection against UVA and UVB radiations, in the short and medium term;
a long-term moisturizing effect.

The interference pigment allows instantaneous optical masking of the red patches of the skin. It is selected in order to transmit a color complementary to red, which will allow rendering of the natural skin color to the areas of the skin affected by red patches. The effect of the composition on the red patches of the skin is therefore immediate.

By means of the titanium dioxide which coats every mica particle, with the interference pigment, it is moreover possible to provide the composition with a protective effect against infrared radiations which, via heating of the skin, promote appearances of rosacea. The interference pigments may therefore also prevent the occurrence or worsening of rosacea or of red patches of the skin.

The interference pigment preferably represents between 0.5% and 10%, preferably between 0.5% and 3% by weight of the composition. The optimum amount of interference pigment providing the composition with good instantaneous masking of the red patches, good protection against infrared radiations and a satisfactory consistence and rendering property, is located between 1% and 3% by weight of the composition.

The interference pigment preferably consists of mica particles coated with titanium dioxide, for which the particle size at 80% is comprised between 10 μm and 60 μm and the DE50 of which is comprised between 18 μm and 25 μm.

Timiron® Super Green marketed by Merck will preferably be used.

The dermatological composition according to the present invention is advantageously free of any other white or colored pigment.

The sunscreen with a broad active spectrum in the UVA and UVB ranges provides the composition with a preventive effect in the short and medium term against the appearance or worsening of red patches of the skin and other outward signs of rosacea. It is actually known that sun damages at the dermis and dermal vessels which are mainly due to UVA are unquestionably involved in the pathogenesis of rosacea.

A mixture of active UVA screens and of active UVB screens will preferably be used, for which the anti-UVA activity is predominant or else screens with a broad spectrum in a sufficient amount so as to obtain consequent UVA protection. Preferably, the amounts of anti-UVA screen and anti-UVB screen in the composition are determined so that the ratio between the protection factor measured by the in vivo test method of the sun protection factor ¹ (SPF) (mainly UVB rays) of the composition and the protection factor measured in vivo by the persistent pigmentation test ² (mainly UVA rays) of the composition, is much less than 3. ¹ a calculation method described in “Méthode internationals d′ essai de facteur de protection solaire (2006)”.² Japan Cosmetic Industry Association (JCIA) Measurement standards for UVA protection efficacy, Nov. 21, 1995.

The photoprotective system preferably represents between 15% and 30% by weight of the composition.

It may be selected from the following organic filters: ethylhexyl salicylate, ethylhexylmethoxy cinnamate, octocrylene, butylmethoxydibenzoyl methane, phenylbenzimidazole-sulfonic acid, n-hexyl 2-(4-diethylamino-2-hydroxybenzoyl)-benzoate, 4-methylbenzylidene-camphor, terephthalidene-dicamphor-sulfonic acid, disodium phenyl dibenzimidazole tetra-sulfonate, 2,4,6-tris-(diisobutyl)-4′-aminobenzalmalonate)-s-triazine, anisotriazine, ethylhexyl triazone, diethylhexylbutamido-triazone, methylene bis-benzotriazolyl-tetramethylbutylphenol, drometrizole trisoloxane, polysilicone-15,1,1-dicarboxy-(2′,2′-dimethyl-propyl)-4,4-diphenylbutadiene, bis-ethylhexylphenol methoxyphenyl triazine.

The sunscreen with a broad UVA active spectrum is preferably selected from Tinosorb® M (methylene bis-benzotriazolyl-tetramethylbutylphenol) and Tinosorb® S (bis-ethylhexyloxyphenol methoxyphenyl triazine), as well as mixtures thereof. Preferably, Tinosorb® M represents between 1% and 14% by weight of the composition and Tinosorb® S represents between 1% and 7% by weight of the composition.

The UVB active sunscreen preferably represents between 1 and 10% by weight of the composition. 2-ethylhexyl 4-methoxycinnamate or broad spectrum filters Tinosorb® M (methylene bis-benzotriazolyl-tetramethylbutylphenol) or, Tinosorb® S (bis-ethylhexyloxyphenolmethoxyphenyl triazine) will be preferably used for their protective aspect in the UVB range.

The dermatological composition according to the present invention moreover comprises one or more soothing and/or moisturizing active ingredients, for a soothing action in the short and medium term and a long-term moisturizing action. The soothing active may be hamamelis water, bisabolol, allantoin, aloe vera, batyl alcohol and beta-glycyrrhetinic acid, hamamelis water will preferably be used. The moisturizing active may be glycerin, d-panthenol, sodium hyaluronate, butylenes glycol, propylene glycol, sorbitol, hyaluronic acid or further chondroitin sulfate acid; glycerin will preferably be used. The composition may also contain other active ingredients, such as anti-oxidants, such as for example α-tocopheryl acetate, ascorbic acid or ascorbyl palmitate.

It may further contain a stabilizer, a sequestering agent and/or anti-radical agent.

The dermatological composition according to the present invention may be prepared as a water-in-oil (W/O) or oil-in-water (O/W) emulsion, a multiple emulsion such as for example a water-in-oil emulsion in water (W/O/W), or an oil-in-water emulsion in oil (O/W/O), or further as a hydrodispersion or lipodispersion, a gel, a stick, or an aerosol.

It preferably appears as an emulsion comprising an oily phase and an aqueous phase, said oily phase containing the sunscreen(s), and said aqueous phase containing the moisturizing and soothing active ingredient(s).

The present invention is illustrated by the following examples.

### 1) Exemplary Composition

Cream for the face, against diffuse red patches and apparent vessels.

| Ingredients | Amount (g) |
|---|---|
| Hamamelis water | 3.00 |
| α-tocopheryl acetate | 0.30 |
| Timiron ® Super Green¹ | 1.00-3.00 |
| Tinosorb ® M² | 4.00-10.00 |
| Tinosorb ® S³ | 1.5-7.00 |
| 2-ethylhexyl 4-methoxycinnamate | 7.00-10.00 |
| 99.5% glycerin | 5.00 |
| Tribehenin | 0.40 |
| C12-C15 alkyl benzoate | 1.00-4.00 |
| Ethylhexyl palmitate | 5.00 |
| Glyceryl stearate | 1.00-2.50 |
| Cyclomethicone | 5.00-8.00 |
| Potassium cetyl phosphate | 1.00-3.00 |
| Hydroxyethyl acrylate | 0.8-2.3 |
| Xanthan gum | 0.1-0.35 |
| Magnesium aluminum silicate | 0.30 |
| Phenoxyethanol | 0.80 |
| Chlorphenesin | 0.30 |
| Benzoic acid | 0.20 |
| Disodium EDTA | 0.10 |
| BHT | 0.01 |
| Water | Qsp 100.00 |

¹Mica coated with titanium oxide, marketed by Merck as Timiron ® Super Green
²Methylenebis-benzotriazolyl-tramethylbutylphenol, marketed by Ciba as Tinosorb ® M
³Bis-ethylhexyloxyphenolmethoxyphenyl triazine, marketed by Ciba as Tinosorb ® S

The cream given as an example is an emulsion of the oil-in-water type, the aqueous phase of which contains hamamelis water, glycerin, EDTA, xanthan gum, magnesium aluminium silicate, chlorphenesin and water and the oily phase of which contains tocopheryl acetate, ethylhexyloxyphenol, methoxycinnamate, diglyceryl behenate, benzoate, ethylhexyl palmitate, glyceryl stearate, potassium cetyl phosphate and siloxane.

### 2) Efficacy Tests

2.2) Evaluation of the Immediate Soothing Effect of the Cream according to Example 1 on a Thermal Simulation Model

The test is conducted on 12 women of 20-42 years of age, on average 30.25 years old.

The experimental program is the following:
making marks with a grease pencil on an area 3 cm×3 cm on the cheek bone,
basal measurement of temperature and of the red patch,
performing thermal stimulation at 38° C. for 6 minutes,
temperature and red patch measurements immediately after simulation (T0),
starting the stopwatch,
depending on randomization, only for the
   application of 20 μL of the cream according to Example 1,
   temperature and red patch measurements at:
T1=3 minutes after thermal stimulation
T2=5 minutes after thermal stimulation
T3=7 minutes after thermal stimulation
T4=10 minutes after thermal stimulation
T5=15 minutes after thermal stimulation
Evaluation of the tolerance of the product,
Performing the same operations on the other cheek,
Evaluation of the tolerance of the product.

Efficacy Results:
skin temperature: after thermal stimulation, the skin temperature is immediately lowered and maintained at the basal value with the cream according to Example 1, unlike the non-treated side for which it is necessary to wait for 10 minutes.
Skin red patch: after thermal stimulation, the skin red patch is significantly improved with the cream according to Example 1 as compared with the non-treated side. Immediately after applying the product, the red patch decreases by 13% versus 3.6% on the non-treated side. A quarter of an hour after applying the product, the red patch has decreased by 14.3% on the side treated with the cream according to Example 1, versus 5% on the non-treated side.

Conclusion: the cream according to Example 1 has a significant effect on the lowering of the temperature on the skin red patch after thermal stimulation.

### 2.2) Consumer Test Carried Out Confidentially

70 women evaluated the cream according to Example 1 over a period of 21 days. After application, the very wide majority of the interviewed women report a flexible and comfortable skin and for more than half of them, this comfort lasts the whole day.

Three quarters of them perceive soothing at the epidermis and two thirds the protective effect of the care product.

The obtained result is estimated as natural by the very wide majority.

At the end of the 5 days of use, three quarter of the questioned women mention less visible red patches (clearly for a quarter of them) and a unified complexion (clearly for almost half of them).

At the end of 21 days of use, the results reported after the first 5 days are confirmed significantly. The red patches are attenuated for the very wide majority and this clearly for half of the women. Complexions are more uniform (84%) and lighter (81%).

### 2.3) Immediate Optical Masking Action of the Red Patches Obtained as a Result of Applying the Cream According to Example 1

The test is conducted on two women having couperose skin and having diffuse red patches on the cheeks.

The starting point of the study (before applying the cream according to Example 1) is noted as T0.

This test is based on the measurement of changes in skin coloration.

Two series of colorimetric measurements are conducted with two different apparatuses:
with a chromameter MINOLTA CR 300® on the one hand;
with a spectrocolorimeter CM508c® on the other hand.

These are apparatuses intended for producing three-dimensional analysis of the color of the skin (hue, saturation, brightness) according to the sensitivity curve of the eye. They indicate the following three parameters in arbitrary units (a.u.):
L*: luminance which corresponds to brightness or luminosity;
a*: represents the hue and saturation of the color on a red-green color axis;
b*: represents the hue and the saturation of the color on a yellow-blue color axis.

Thus, in the case of a change in the color:
if the color of the skin is lighter: the parameter L* increases,
if the skin is less red: the parameter a* decreases;
if the skin is less “yellow” (a less tanned aspect): the parameter b* decreases.

### Procedure:

Measurement area: skin coloration is measured at the cheek on an area delimited beforehand and marked with a marking mask. The diameter of the measurement surface is 1 cm.
Applying the cream to be tested: the cream according to Example 1 is applied in an amount of 2 mg/cm² over a wider surface (about 5 cm×5 cm) than the actual measurement area (probe diameter).
Measurement time: the values were measured at T0 (before application of the cream) and at T10 (after having laid it for 10 minutes). Each measurement is repeated three times.

### Results:

The results of the measurements carried out on each subject are shown in the Tables 1 and 2 hereafter:

TABLE 1
Measurements conducted with the
chromameter of the parameters L*, a* and b* before and
just after applying the cream according to Example 1 on
skins exhibiting red patches.

| | Subject A | | | Subject B | | |
|---|---|---|---|---|---|---|
| | | | Change | | | Change |
| | Av- | Average | in the | | Average | in the |
| Parameters | erage | at | averages | Average | at | averages |
| in a.u. | at T0 | T10 | (%) | at T0 | T10 | (%) |
| L* | 59.32 | 60.86 | +2.59% | 64.85 | 64.93 | +0.11% |
| a* | 18.35 | 15.62 | −14.86% | 14.82 | 13.80 | −6.84% |
| b* | 13.02 | 10.35 | −20.53% | 16.66 | 12.49 | −25.07% |

TABLE 2
measurements conducted with the
spectrocolorimeter of the parameters L*, a* and b*
before and just after applying the cream according to
Example 1 on skins exhibiting red patches

| | Subject A | | | Subject B | | |
|---|---|---|---|---|---|---|
| | | | Change | | | Change |
| | Av- | Average | in the | | Average | in the |
| Parameters | erage | at | averages | Average | at | averages |
| in a.u. | at T0 | T10 | (%) | at T0 | T10 | (%) |
| L* | 56.60 | 59.77 | +5.53% | 62.72 | 65.61 | +4.60% |
| a* | 17.24 | 13.88 | −19.45% | 14.84 | 12.07 | −18.65% |
| b* | 13.55 | 8.93 | −34.09% | 15.97 | 12.04 | −24.57% |

It is observed that:
L* increases: meaning that there is lightening of the skin globally.
a* decreases: which reasonably expresses that there is a reduction of redness globally.
b*: decreases: there is also a trend of reducing the tanned aspect of the skin.

The Applicant has demonstrated the immediate action of a composition according to the present invention on red patches. Indeed, after only 10 minutes of application, the skin is visually lighter and less red.

1. A dermatological composition for preventing and/or treating rosacea, couperose or skins which exhibit diffuse red patches or small dilated vessels, characterized in that it contains:
at least one interference pigment comprising mica as particles coated with titanium dioxide, transmitting a color complementary to that of red and for which the particle size at 80% is comprised between 10 μm and 60 μm and the DE50 of which is comprised between 18 μm and 25 μm
at least one sunscreen with a broad active spectrum in the UVA and UVB ranges;
one or more soothing and/or moisturizing active ingredients; and
the balance as dermatologically acceptable excipient(s) required for formulating said composition.

2. The dermatological composition according to claim 1, characterized in that it is provided with a triple action staged over time.

3. The dermatological composition according to claim 2, characterized in that said triple action staged over time is broken down into:
an immediate optical masking action on the red patches;
a soothing effect and/or protection against UVA and UVB radiations, in the short and medium term;
a long-term moisturizing effect.

4. The dermatological composition according to any of claims 1 to 3, characterized in that it contains about between 0.5% and 10%, preferably about between 0.5% and 3% by weight of said interference pigment.

5. The dermatological composition according to claim 1, characterized in that the ratio between the protection factor as measured by the in vivo test method of the sun protection factor of the composition and the protection factor measured in vivo by testing persistent pigmentation of the composition, is less than 3.

6. The dermatological composition according to claim 1, characterized in that said UVA active screen is selected from Tinosorb® M and Tinosorb® S, as well as from mixtures thereof.

7. The dermatological composition according to claim 1, characterized in that it contains about between 1% and 10% by weight of an UVB active sunscreen.

8. The dermatological composition according to claim 7, characterized in that said UVB active sunscreen is 2-ethylhexyl 4-methoxycinnamate.

9. The dermatological composition according to claim 1, characterized in that the active ingredients are selected from hamamelis water, glycerin and α-tocopheryl acetate.

10. The dermatological composition according to claim 1, characterized in that it further contains a stabilizer, a sequestering agent and/or an anti-radical agent.

11. The dermatological composition according to claim 1, characterized in that it contains an emulsion comprising an oily phase and an aqueous phase, said oily phase containing the sunscreen, and said aqueous phase containing the active ingredient(s).

12. The dermatological composition according to claim 11, characterized in that the emulsion is an oil-in-water emulsion.

13. The dermatological composition according to claim 1, characterized in that it fits at least substantially the following composition:

| Ingredients | Amount (g) |
|---|---|
| Hamamelis water | 3.00 |
| α-tocopheryl acetate | 0.30 |
| Timiron ® Super Green¹ | 1.00-3.00 |
| Tinosorb ® M² | 4.00-10.00 |
| Tinosorb ® S³ | 1.5-7.00 |
| 2-ethylhexyl 4-methoxycinnamate | 7.00-10.00 |
| 99.5% glycerin | 5.00 |
| Tribehenin | 0.40 |
| C12-C15 alkyl benzoate | 1.00-4.00 |
| Ethylhexyl palmitate | 5.00 |
| Glyceryl stearate | 1.00-2.50 |
| Cyclomethicone | 5.00-8.00 |
| Potassium cetyl phosphate | 1.00-3.00 |
| Hydroxyethyl acrylate | 0.8-2.3 |
| Xanthan gum | 0.1-0.35 |
| Magnesium aluminum silicate | 0.30 |
| Phenoxyethanol | 0.80 |
| Chlorphenesin | 0.30 |
| Benzoic acid | 0.20 |
| Disodium EDTA | 0.10 |
| BHT | 0.01 |
| Water | Qsp 100.00 |
